(19) United States
(12) Patent Application Publication
(10) Pub. No.: US 2009/0069278 A1
(43) Pub. Date: Mar. 12, 2009

(54) Hormonally-timed dermatological preparations

(76) Inventor: Frances J. R. Richmond, South Pasadena, CA (US)

Correspondence Address: Chiaoyun Kuo;Agent for Applicants, 18691 Paseo Cortez, Irvine, CA, 92606, US

(21) Appl. No.: 12/228,063

(22) Filed: Aug. 1, 2005

(51) Int. Cl. A61K 31/57; A61P 17/00

(52) U.S. Cl. 514/177

(57) ABSTRACT

This invention provides a dermatological preparation to improve skin cosmesis or conditioning by using different formulations in a sequence that suits the needs of the skin at different points of the hormonal cycle. In its simplest implementation, this would be a two-phase skin treatment, such as a cosmetic, lotion or soap whose formulation changes to balance the differing states of hydration and reactivity of the skin during the early and late phases of the cycle. The formulations would be made in a way to be easily identified and delivered conveniently for use at different times of a hormonal cycle. The preparation would be packaged to permit the convenient purchase and dispensing of the different formulations in a manner that could be easily sequenced with the hormonal cycle.

### 1. RELATED APPLICATIONS

This application claims priority of U.S. Provisional Application Ser. No. 60/704,296 filed Aug. 1, 2005, which is incorporated herein by reference in its entirety.

### 2. BACKGROUND

2.1 Field of the Invention

The present invention relates to dermatological preparations, and more particularly, to hormonally-timed dermatological preparations.

2.2 Description of the Related Art

Hormones are well-known to affect the condition of the skin. When these hormonal balances shift, dermatological properties shift as well. In women, menstrual cycling is associated with changes in skin thickness, water retention, blood flow and immune reactivity (Agner, Damm, and Skouby, J. Am. Acad. Dermatol. 24(4):566-70 (1991); Harvell, Hussna-Saeed, and Maibach, Contact Dermatitis 27(5):294-301(1992); Eisenbeiss, Welzel, and Schmeller, Br. J Dermatol 139(3):462-7 (1998); Kirmaz, Yuksel, Mete, Bayrak and Baytur, Asian Pac. J Allergy Immunol. 22(4):197-203 2004), as the result of increasing levels first of estrogen and then of progesterone secretion. Skin reactions to these shifts are most apparent in a small proportion of women who have frank hypersensitivities to progesterone. These women have obvious dermatological and immunogenic problems during the immediate premenstrual period, that include skin rashing, acne and hyperpigmentation (Iteskson, Lazarov, Cordoba, Zeitune, Abraham, and Seidman, J. Reprod. Med. 49(3): 195-9 (2004)). As might be anticipated, special cosmetic or therapeutic interventions are often recommended for such serious skin reactions, typically during the period when progesterone is secreted in high concentrations. Evidence of the important role played by cycling hormones is also apparent from the effects of contraceptive hormones on the skin of premenopausal women. The use of contraceptive hormones is often recommended to young women to stabilize hormone levels because this is associated with a reduction in acne (Cibula, Paseka, Unzeitig, Horejsi, Rotta, and Chroust, Ceska Gynekol. 65(2):79-82 (2000)). Loss of cyclical hormone production at menopause is associated with thinning of the skin, decreased collagen and sebum production and reduced healing capacity (Shah and Maibach, Am. J. Clin. Dermatol. 2(3):143-50 (2001)). These conditions can be mitigated by hormone replacement therapies. Some women, in fact, choose to continue with hormonal therapy to control the symptoms of menopause and enhance the appearance of their skin despite concerns about cardiac or other complications. Commonly, replacement therapies attempt to mimic the normal cycling of the hormones by first supplementing estrogen, and then later adding progesterone to replicate the patterns of hormonal cycling typical of the menstrual pattern. However, the less pathologic skin conditions that do not require drug therapy but are present widely in the general population have not received much attention when developing skin treatments, lotions, cosmetics and soaps.

A knowledge of the physiological effects that normal or imposed hormonal cycling has on the skin may be used to improve the formulation and timing of dermatological products most suitable for the skin at different points in the cycle. Many products have been developed to enhance skin properties or appearance, but these are marketed for use at all time points throughout a monthly cycle. Typically a woman will buy a single facial moisturizer or soap that is used continuously, even when the woman is dissatisfied with the product for some of the time. There is a failure to appreciate that different products might be needed at different points in the menstrual cycle. This often leads women on an expensive search for a better cosmetic or cleansing product, but this search is handicapped by the fact that each product may work best for only one part of the cycle. What is needed, and what is claimed here, is a set of dermatological preparations whose formulations are specialized for administration at different times during the hormonal cycle. A plurality of such preparations would be packaged so that the customer can be reminded to use different formulations appropriate to different parts of the hormonal cycle.

### 3. SUMMARY OF THE INVENTION

It is an object of this invention to improve skin cosmesis or conditioning by using different formulations of the dermatological preparation in a sequence that suits the needs of the skin at different points of the hormonal cycle. In its simplest implementation, this would be a two-phase skin treatment, such as a cosmetic, lotion or soap whose formulation changes to balance the differing states of hydration and reactivity of the skin during the early and late phases of the cycle.

It is also an object of this invention to facilitate improvements to the appearance or condition of the skin by ensuring that a plurality of formulations can be identified and delivered conveniently for use at different times of a hormonal cycle.

It is a further object of this invention to teach a method by which such a product could be packaged to permit the convenient purchase and dispensing of the different formulations in a manner that could be easily sequenced with the hormonal cycle.

### 4. BRIEF DESCRIPTION OF THE DRAWINGS

FIG. 1 illustrates the basic premise of the present invention.

FIG. 2 illustrates one embodiment of the present invention.

FIG. 3 illustrates another embodiment of the present invention.

### 5. DETAILED DESCRIPTION OF THE INVENTION

In general, the present disclosure describes hormonally-timed dermatological preparations. For easier understanding, specific embodiments of present invention are shown in the drawings. However, the present invention is not limited to the example embodiments describe below.

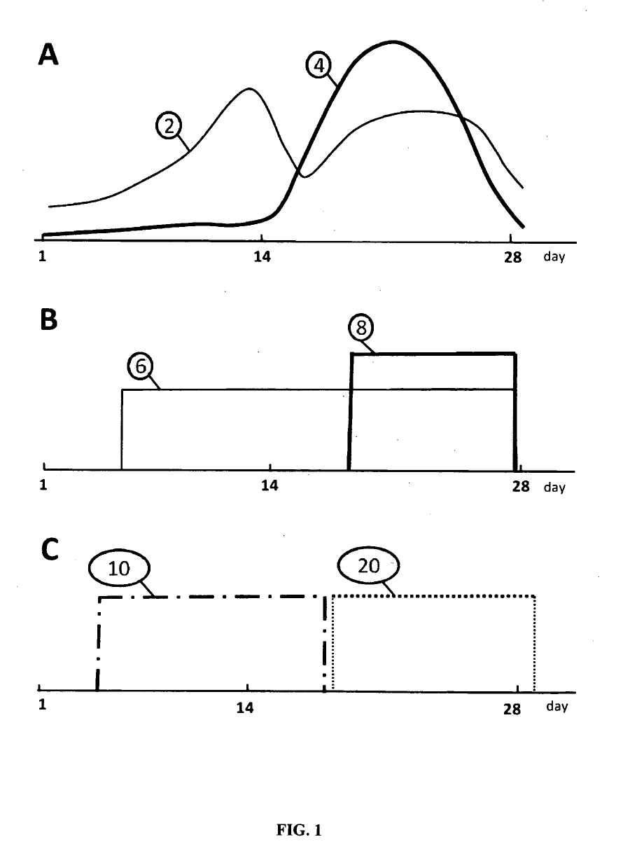

Methods to time, produce and package a sequence of dermatological formulations is described in FIGS. 1, 2 and 3. FIG. 1 illustrates the basic premise of the invention. It has three panels that illustrate, in panel A, the pattern of cycling of estrogen 2 and progesterone 4 in a normal menstrual period of 28 days. Below, in B, is illustrated a common regimen of hormone replacement therapy for a postmenopausal woman in which conjugated estrogens are administered from day 4 to day 18 after menstruation begins 6, and then progesterone is administered together with the estrogen for the last 10 days of the cycle 8. Panel C of FIG. 1 shows the simplest embodiment of the invention in which two formulations are supplied, a first formulation 10 designed to match the needs of the skin during the estrogen phase and a second formulation 20 for use during the phase when progesterone is also taken. The actual number and composition of formulations could vary depending upon the target user. In one embodiment of the method, the formulation supplied to a post menopausal woman on hormone replacement therapy would vary in fat content from one part of the cycle to the next. During the early phase, when estrogen administration enhances hydration and thickening of the skin, and when the skin is faster to heal, the first formulation 10 would be lighter and less oily. During the second part of the cycle, when progesterone exerts a braking effect on the estrogenic effects, the second formulation 20 would contain more fat or humectants to protect the skin and to increase its state of hydration. A simple pair of exemplary preparations is shown in Table I below, but many such combinations could be used to achieve the required effect.

TABLE 1

| | Formulation 1 | Formulation 2 |
|---|---|---|
| Almond oil | 2.5 | 2.5 |
| Cetyl Alcohol | 0.8 | 0.8 |
| Stearic acid | 20 | 20 |
| Triethanolamine | 1.8 | 1.8 |
| Water | 69.5 | 68 |
| Glycerol | 5 | 5 |
| Preservative | 0.2 | 0.2 |
| Perfume | 0.2 | 0.2 |
| Lanolin | | 1.5 |

In another embodiment designed for younger women or women with sensitivity to progesterone, the second formulation 20 administered during the late phase of the cycle, when acne is particularly problematic, would differ from the first formulation 10 by the addition of constituents to control sebum production, reduce bacterial proliferation and inflammation, or soothe or disguise rashing. By supplying two or more sequenced formulations, better control of skin properties may be achieved with greater convenience to the user. It would be obvious to one normally skilled in the art that this invention could be adapted for situations in which the timing of hormones varied, or in which the formulations were altered for specific types of skin and different skin conditions that are affected by hormonal cycling.

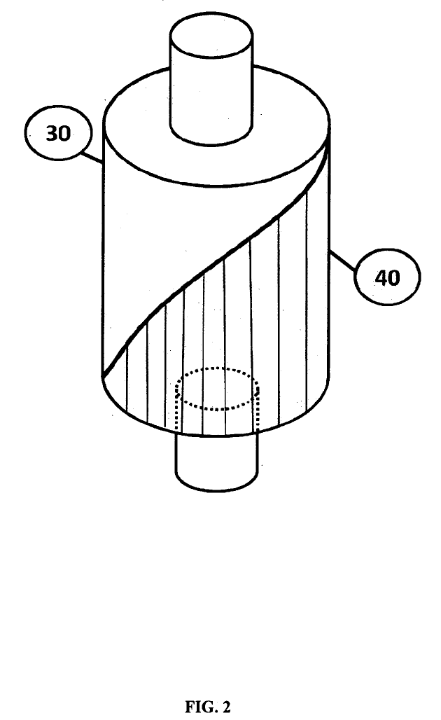

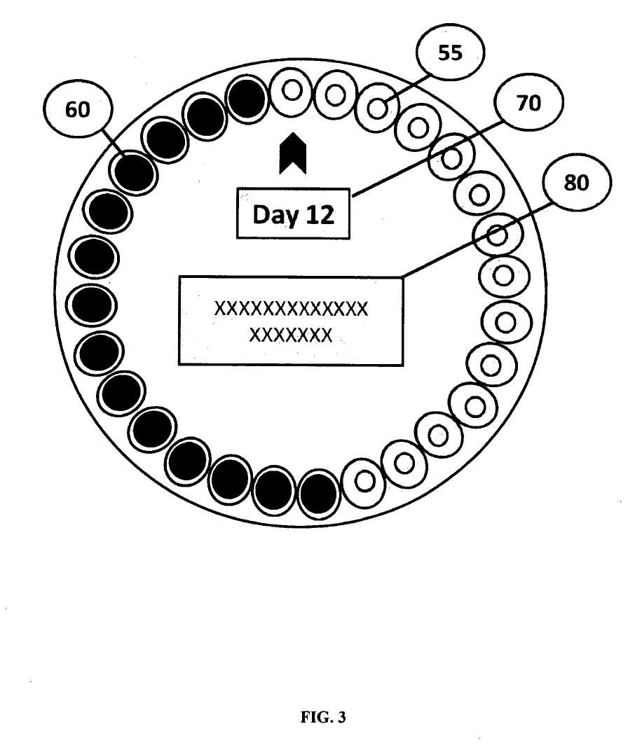

The invention includes means to deliver combinations of formulations. The preferred embodiment has the basic form of a package containing a plurality of reservoirs containing more than one dermatological formulation, and a method of labeling that identifies which formulations are to be used at specific points in the menstrual cycle. FIGS. 2 and 3 illustrate two exemplary packages that could be used to ensure that supplied formulations are used in accordance with cycled exogenous hormones or medications. FIG. 2 shows the simplest embodiment in which a pair of fused tubes 30, 40 are differentiated by color or other labeling means. Each is filled with a different dermatological formulation. In this embodiment, appropriate for example, for women who receive hormone replacement therapy in the form of two bottles of pills, the instruction could be to use the contents of one tube during the time when only estrogen pills are taken, and use the product on the second tube when both estrogen and progesterone pills are taken together. In the embodiment illustrated in FIG. 3, the formulations are packaged individually in a blister pack or other such means that have separate reservoirs 55 containing a quantity of material adequate for a single session or day. The embodiment in FIG. 3 shows the individual applications in softgel capsules 50, 60 arranged circumferentially in a package reminiscent of a familiar birth control dispenser, that provides product for a single month. Different types of product are differentiated by marking means such as color, location or other labeling 70. In this embodiment, the white capsules 55 are used for the first part of the cycle whereas the dark capsules 60 are used for the second part of the cycle. Additional labeling is needed 80 to identify the product further and provide instructions for safe use. It would be obvious to one skilled in the art that other types of packaging could be employed to practice this method. Further, this method could be adapted easily to dispense two or more formulations appropriate for use in women who experience endogenous hormonal cycling after puberty and prior to menopause. Alternative forms of packaging to practice this invention would be obvious to one normally skilled in the art. The examples provided herein are intended only to illustrate the invention and not to limit its scope.

What is claimed is:

1. A dermatological preparation, comprising at least one pharmaceutical formulation to be administered according to the phase of a menstrual cycle.

2. The dermatological preparation according to claim 1, wherein said at least one pharmaceutical formulation exhibits a water composition of at least 50%.

3. The dermatological preparation according to claim 1, wherein said at least one pharmaceutical formulation exhibits a humectants composition of at least 20%.

4. The dermatological preparation according to claim 1, wherein said at least one pharmaceutical formulation exhibits a sebum content control agent composition of at least 0.5%.

5. The dermatological preparation according to claim 1, wherein said at least one pharmaceutical formulation contains constituents to control bacterial proliferation, to control inflammation, to control rash, or combination thereof.

6. A dermatological preparation comprising at least two pharmaceutical formulations to be administered during different phases of a menstrual cycle, where said at least two pharmaceutical formulations differ in their chemical composition in order to suit the different needs of the user at different phases of the menstrual cycle.

7. The dermatological preparation according to claim 6, wherein the difference between said at least two pharmaceutical formulations includes a difference in water content.

8. The dermatological preparation according to claim 6, wherein the difference between said at least two pharmaceutical formulations includes a difference in humectants content.

9. The dermatological preparation according to claim 6, wherein the difference between said at least two pharmaceutical formulations includes a difference in drying agents.

10. The dermatological preparation according to claim 6, wherein said difference between the at least two pharmaceutical formulations includes a difference in antibiotic content.

11. A dermatological preparation consisting of at least two different pharmaceutical formulation for administration during different phases of hormone replacement therapy in post-menopausal women.

12. The dermatological preparation according to claims 1, 6 or 11, wherein said dermatological preparation is in form of soap, cosmetic, or lotion.

13. The dermatological preparation according to claims 6 or 11, wherein said dermatological preparation is delivered in a package that designates the phase during which each different pharmaceutical formulation should be used.
